(19) United States
(12) Patent Application Publication
(10) Pub. No.: US 2026/0157788 A1
(43) Pub. Date: Jun. 11, 2026

(54) RADIOFREQUENCY GENERATOR WITH AUTOMATED OPERATIONAL MODES

(71) Applicant: Relievant Medsystems, Inc., Edina, MN (US)

(72) Inventors: Raul Serrano Carmona, Los Angeles, CA (US); Alexander Pruitt, San Jose, CA (US); Jin Yu, Valley Village, CA (US); James A. Sievert, Bradenton, FL (US); Archana Kumar, Corcoran, MN (US)

(21) Appl. No.: 19/414,042

(22) Filed: Dec. 9, 2025

Related U.S. Application Data
(60) Provisional application No. 63/729,881, filed on Dec. 9, 2024.

(51) Int. Cl. A61B 18/14; A61B 17/00; A61B 18/00

(52) U.S. Cl. CPC A61B 18/14; A61B 2017/00199; A61B 2018/00577; A61B 2018/00702; A61B 2018/00791; A61B 2018/00875

(57) ABSTRACT

A tissue ablation system employing a radiofrequency generator and a radiofrequency probe, where a generator controller can be employed to automate selected operational modes of the generator. The operational modes can include determining if the probe is connected to the generator, determining the location of the probe, and determining an impedance of the probe. The generator can also display multiple channels on a display element, and the controller can generate summary information of the ablation procedure, including information associated with probe impedance and temperature and the power supplied by the generator.

### CROSS REFERENCE TO RELATED APPLICATION

The present application claims priority to U.S. Provisional Patent Application No. 63/729,881, filed Dec. 9, 2024, the disclosure of which is incorporated herein in its entirety.

### BACKGROUND OF THE INVENTION

Radiofrequency (RF) generators are widely used in medical ablation procedures to treat various medical conditions, including for example cardiac arrhythmias, chronic pain, and cancerous tumors. The RF generators typically are configured to deliver controlled RF energy to specific target tissue areas, causing localized heating and tissue necrosis. The effectiveness of RF ablation procedures depends on the precise delivery of energy, requiring close monitoring and control by the operator. Conventional RF generators typically operate by producing alternating electrical currents that can be delivered to the target tissue via electrodes on associated RF probes, generating resistive heating through the tissue's natural impedance. Feedback mechanisms, including temperature sensors or impedance monitoring, allow the system to adjust the power output to maintain optimal energy delivery during the procedure.

The conventional RF generator can employ a display element that can display a series of user interfaces that are designed to provide real-time feedback on selected operational parameters, such as power output, impedance, temperature, and ablation time. The user interfaces often display segmented digital readouts or basic graphical elements. Buttons, dials, or touchscreens can enable operators to set and adjust parameters as needed. Despite their functionality, the traditional user interfaces can be limited in intuitiveness and clarity, potentially increasing the cognitive load on the user during complex surgical procedures.

### SUMMARY OF THE INVENTION

The tissue ablation system employing a radiofrequency generator of the present invention can generate user interfaces that can be displayed on a display element that offer improved visualization of key operational parameters and metrics and more intuitive control mechanisms. The present invention addresses the limitations of conventional radiofrequency generators by introducing novel systems and methods for displaying information and interacting with the radiofrequency generator, optimizing usability and procedural outcomes.

The tissue ablation system of the present invention includes a radiofrequency generator having a controller having memory and a processor, and first and second radiofrequency probes. The processor of the controller can be programmed to automatically perform a series of operational functions, in sequence, without operator prompting or interruption. The operational functions can include determining if the first radiofrequency probe is electrically coupled to the radiofrequency generator, determining a type of arrangement of the first radiofrequency probe, determining a placement position of the first radiofrequency probe, and determining a probe impedance of the first radiofrequency probe to determine if the at least one electrode is exposed to tissue. The operator of the tissue ablation system can then select a duration of the ablation procedure with the first radiofrequency probe, and if desired a duration of an ablation procedure with the second radiofrequency probe. The system can then perform a warm-up procedure to warm up the first and second radiofrequency probes. The ablation procedure can be initiated by supplying suitable radiofrequency power to the radiofrequency probes, and then display a time remaining in the ablation procedure associated with the first and optionally the second radiofrequency probe. Upon completion of the ablation procedure with the radiofrequency probes, the RF generator can display on a display element a summary of selected parameters associated with the first and second radiofrequency probes (e.g., channel 1 and channel 2). The selected parameters can include an impedance of the radiofrequency probes during the ablation procedure, a temperature of the radiofrequency probes during the ablation procedure, and a power supplied by the power source to the radiofrequency probes during the ablation procedure.

The present inventio is directed to a tissue ablation system comprising a radiofrequency generator and at least a first radiofrequency probe. The radiofrequency generator has a power source, a controller operatively coupled to the power source and having a processor and a memory element, a plurality of connection ports, and a display element. The first radiofrequency probe is electrically coupled to the radiofrequency generator to form a first channel and has at least one first electrode and a first temperature sensor. The power source can deliver radiofrequency power to the first radiofrequency probe sufficient to provide controlled heating of tissue of a patient surrounding the first electrode during an ablation procedure. The processor can be configured to execute program instructions stored in the memory element such that, when executed, cause the processor to (a) determine if the first radiofrequency probe is electrically coupled to the radiofrequency generator, (b) determine a type of electrode arrangement of the first radiofrequency probe, (c) determine a placement position of the first radiofrequency probe relative to the patient, and/or (d) determine a probe impedance of the first radiofrequency probe to determine if the at least one first electrode is exposed to the tissue. The system can also (e) based on user selection data, determine a duration of the ablation procedure with the first radiofrequency probe, (f) perform a warm-up procedure to warm up the first radiofrequency probe, (g) initiate the ablation procedure by supplying power to the first radiofrequency probe, (h) display a time remaining in the ablation procedure with the first radiofrequency probe on the display element, and (i) upon completion of the ablation procedure with the first radiofrequency probe, display on the display element a summary of selected parameters associated with the first radiofrequency probe. The selected parameters can include an impedance of the first radiofrequency probe during the ablation procedure, a temperature of the first radiofrequency probe during the ablation procedure, and a power supplied by the power source to the first radiofrequency probe during the ablation procedure.

The processor can also be configured to automatically perform any combination of the (a)-(d) operational modes. According to one embodiment, in connection with the first probe, the processor is configured to perform two or more of (a)-(d), all of (a)-(d), or all of (a)-(d) in sequence. The processor can be further configured to determine the placement position of the first radiofrequency probe by processing temperature data from the first temperature sensor and comparing the temperature data to a threshold temperature to determine probe location. The processor can also be configured to determine a probe impedance of the first radiofrequency probe by comparing the probe impedance with a threshold probe impedance value and then determine that the electrode is exposed to the tissue when the probe impedance is below the threshold probe impedance value.

The tissue ablation system can also include a second radiofrequency probe that is electrically coupled to the radiofrequency generator to form a second channel and having at least one second electrode and a second temperature sensor. The processor is further configured to independently, or in combination with the first probe, (a) determine if the second radiofrequency probe is electrically coupled to the radiofrequency generator, (b) determine a type of electrode arrangement of the second radiofrequency probe, (c) determine a placement position of the second radiofrequency probe relative to the patient, and/or (d) determine a probe impedance of the second radiofrequency probe to determine if the at least one second electrode is exposed to the tissue of the patient. The processor is further configured to, based on user selection data, determine a duration of the ablation procedure with the second radiofrequency probe, perform a warm-up procedure to warm up the second radiofrequency probe, initiate the ablation procedure by supplying power to the second radiofrequency probe, display a time remaining in the ablation procedure with the second radiofrequency probe on the display element, and/or upon completion of the ablation procedure with the second radiofrequency probe, display a summary of selected parameters associated with the second radiofrequency probe. The selected parameters include an impedance of the second radiofrequency probe during the ablation procedure, a temperature of the second radiofrequency probe during the ablation procedure, and the power supplied by the power source to the second radiofrequency probe during the ablation procedure,

The processor can also be configured to automatically perform any combination of the (a)-(d) operational modes. According to one embodiment, the processor is configured to perform, in connection with the second radiofrequency probe, two or more of (a)-(d), all of (a)-(d), or all of (a)-(d) in sequence. The processor can be further configured to determine the placement position of the second radiofrequency probe by processing temperature data from the second temperature sensor and comparing the temperature data to a threshold temperature to determine probe placement.

The processor is further configured to display information associated with the first channel and the second channel on the display device, and/or display information associated with the first channel and the second channel individually or simultaneously on the display device. The plurality of connector ports comprises at least one ground connector port and a plurality of probe connector ports.

The present invention is further directed to a computer-implemented method of ablating tissue in an ablation procedure using a tissue ablation system. The tissue ablation system includes a radiofrequency generator and a first radiofrequency probe. The radiofrequency generator has a power source, a controller operatively coupled to the power source having a processor and a memory element, a plurality of connection ports, and a display element. The first radiofrequency probe is electrically coupled to the radiofrequency generator to form a first channel and has at least one first electrode and a first temperature sensor. The power source is configured to deliver radiofrequency power to the first electrode of the first radiofrequency probe to provide controlled heating of the tissue of the patient surrounding the first electrode during an ablation procedure. The processor can also be configured to execute program instructions stored in the memory element such that, when executed, cause the processor to (a) determine if the first radiofrequency probe is electrically coupled to the radiofrequency generator, (b) determine a type of electrode arrangement of the first radiofrequency probe, (c) determine a placement position of the first radiofrequency probe relative to the patient, (d) determine a probe impedance of the first radiofrequency probe to determine if the at least one first electrode is exposed to the tissue, Further, based on the user selection data, the processor can be further configured to (e) determine a duration of the ablation procedure with the first radiofrequency probe, (f) perform a warm-up procedure to warm up the first radiofrequency probe, (g) initiate the ablation procedure by supplying power to the first radiofrequency probe, (h)display a time remaining in the ablation procedure with the first radiofrequency probe on the display element, and (i) upon completion of the ablation procedure with the first radiofrequency probe, display on the display element a summary of selected parameters associated with the first radiofrequency probe. The selected parameters can include an impedance of the first radiofrequency probe during the ablation procedure, a temperature of the first radiofrequency probe during the ablation procedure, and a power supplied by the power source to the first radiofrequency probe during the ablation procedure.

The processor can also be configured to automatically perform any combination of the (a)-(d) operational modes. According to one embodiment, in connection with the first radiofrequency probe, the processor is configured to perform two or more of (a)-(d), all of (a)-(d), or all of (a)-(d) in sequence. The processor can be further configured to determine the placement position of the first radiofrequency probe by processing temperature data from the first temperature sensor and comparing the temperature data to a threshold temperature to determine probe location. The processor can also be configured to determine a probe impedance of the first radiofrequency probe by comparing the probe impedance with a threshold probe impedance value and then determine that the electrode is exposed to the tissue when the probe impedance is below the threshold probe impedance value.

The method of the present invention can also include determining the placement position of the first radiofrequency probe by processing temperature data from the first temperature sensor and comparing the temperature data to a threshold temperature to determine probe location. The method can also include configuring the processor to determine a probe impedance of the first radiofrequency probe by comparing the probe impedance with a threshold probe impedance value and then determining that the first electrode is exposed to the tissue when the probe impedance is below the threshold probe impedance value.

The method of the present invention can also employ a second radiofrequency probe that is electrically coupled to the radiofrequency generator to form a second channel and having at least one second electrode and a second temperature sensor. The method can include (a) determining if the second radiofrequency probe is electrically coupled to the radiofrequency generator, (b) determining a type of electrode arrangement of the second radiofrequency probe, (c) determining a placement position of the second radiofrequency probe relative to the patient, and (d) determining a probe impedance of the second radiofrequency probe to determine if the at least one second electrode is exposed to the tissue of the patient. The method can also include, based on user selection data, determining a duration of the ablation procedure with the second radiofrequency probe, performing a warm-up procedure to warm up the second radiofrequency probe, initiating the ablation procedure by supplying power to the second radiofrequency probe, displaying a time remaining in the ablation procedure with the second radiofrequency probe on the display element, and/or, upon completion of the ablation procedure with the second radiofrequency probe, display a summary of selected parameters associated with the second radiofrequency probe. The selected parameters can include an impedance of the second radiofrequency probe during the ablation procedure, a temperature of the second radiofrequency probe during the ablation procedure, and the power supplied by the power source to the second radiofrequency probe during the ablation procedure.

The processor can also be configured to automatically perform any combination of the (a)-(d) operational modes. According to one embodiment, in connection with the second probe, the processor can be configured to perform two or more of (a)-(d), all of (a)-(d), or all of (a)-(d) in sequence. The processor can be further configured to determine the placement position of the second radiofrequency probe by processing temperature data from the second temperature sensor and comparing the temperature data to a threshold temperature to determine probe location. The processor can also be configured to determine a probe impedance of the second radiofrequency probe by comparing the probe impedance with a threshold probe impedance value and then determining that the second electrode is exposed to the tissue when the probe impedance is below the threshold probe impedance value.

The method can further include configuring the processor to display information associated with the first channel and the second channel on the display device. The information can be displayed individually or simultaneously on the display device.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic depiction of a tissue ablation system according to the teachings of the present invention.

FIGS. 2 and 3 are schematic flow chart diagrams illustrating the operational steps performed by the radiofrequency generator of the tissue ablation system of FIG. 1.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION OF THE INVENTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The term “application” or “software application” or “program” as used herein is intended to include or designate any type of procedural software application and associated software code which can be called or can call other such procedural calls or that can communicate with a user interface or access a data store. The software application can also include called functions, procedures, and/or methods.

The term “graphical user interface” or “user interface” as used herein refers to any software application or program, which is used to present data to an operator or end user via any selected hardware device, including a display screen, or which is used to acquire data from an operator or end user for display on the display screen. The interface can be a series or system of interactive visual components that can be executed by suitable software. The user interface can hence include screens, windows, frames, panes, forms, reports, pages, buttons, icons, objects, menus, tab elements, and other types of graphical elements that convey or display information, execute commands, and represent actions that can be taken by the user. The objects can remain static or can change or vary when the user interacts with them.

A radiofrequency ablation (RFA) procedure is a minimally invasive medical procedure that is used to ablate targeted tissue at a surgical site, and typically involves using one or more energy delivery devices (e.g., radiofrequency (RF) probes) having one or more electrodes associated therewith. The radiofrequency probe can be coupled to one or more radiofrequency (RF) generators to deliver power in the form of radiofrequency energy to the targeted tissue via the electrodes. The radiofrequency energy generates localized heat which is used to target and destroy (e.g., ablate) the targeted tissue. In pain management, the targeted tissue can be nerve tissue responsible for sending pain signals.

The RFA procedure can be utilized to ablate different types of tissue, such as nerve tissue, adipose tissue, muscle tissue, and the like. The RFA procedure can be performed at various locations on an external surface of a subject or patient or within the subject. The RFA procedure performed within the subject can be performed in a minimally invasive manner (e.g., via a percutaneous, laparoscopic, endoscopic, or intravascular approach) or in a more invasive manner (e.g., via an open surgical procedure). For example, Applicant's existing technology (the Intracept® procedure by Relievant® Medsystems, Inc.) offers a safe and effective minimally invasive procedure that targets the basivertebral nerve (BSN) for the relief of chronic low back pain that originates at least partly from one or more endplates of one or more vertebral bodies. The Intracept® procedure involves application of radiofrequency energy from a radiofrequency generator using a bipolar radiofrequency probe, where the applied radiofrequency energy is sufficient to ablate the basivertebral nerve within the vertebral body. The procedure can be performed in multiple different vertebral bodies sequentially or simultaneously using a single radiofrequency probe or using multiple radiofrequency probes coupled to a single generator. The basivertebral nerve trunk can then be ablated by the RF probe. Other intraosseous nerves within the vertebral body that innervate the endplates and/or intervertebral disc can also be targeted and ablated.

The RFA procedure is typically performed using a surgical introducing or access assembly that can include a hollow cannula that forms a sheath and a stylet that is disposed within the cannula until a distal tip of the stylet extends beyond the cannula. The stylet adds rigidity and support to the cannula when inserted into the patient. The stylet helps the surgeon guide the assembly to the surgical site. Once the cannula is in position at the surgical site, then the stylet is removed from the cannula and the RF probe is inserted therein. The RF probe can then deliver RF energy to the targeted tissue at the surgical site. In a bipolar RF probe arrangement, the probe can include a pair of electrodes, including a first active energy delivering electrode and a second return electrode. According to another arrangement, the RF probes can be configured in a monopolar arrangement, where an RF probe employs an active energy delivering electrode and a separate device or element can be used as a return. The return can be configured as a return pad coupled to the subject or a return electrode disposed on a separate RF probe. For example, a first RF probe can be used that includes the active energy delivering electrode and a second RF probe can be used that includes a return electrode. The RF probe can also include additional structure, including for example temperature sensors (e.g., thermocouples or thermistors), that can be employed to sense or detect the temperature at the surgical site during the surgical procedure. The sensed or detected temperature can provide important information to the surgeon about the ablation procedure. The RFA procedure contemplates positioning the two electrodes of the one or more radiofrequency probes within the vertebral body. The RF generator supplies power to the active electrode of the RF probe at a selected power or voltage level and for a time sufficient to create a desired lesion within the vertebral body sufficient to ablate the basivertebral nerve within the vertebral body. In some implementations, the step of applying power to the RF probe includes supplying power for a selected duration or period of time (e.g., 1 minute to 1 hour or for any time within this range) to achieve a selected temperature at the surgical site. The temperature of the thermal energy supplied by the RF probe to the targeted tissue can range from between about 50° C. and about 115° C. (e.g., from about 70° C. to about 90° C., from about 75° C. to about 90° C., from about 83° C. to about 87° C., from about 80° C. to about 100° C., from about 85° C. to about 95° C., from about 90° C. to about 110° C., from about 95° C. to about 115° C., or overlapping ranges thereof). The temperature ramp-up during the surgical procedure can range from between about 0.1-5 degrees Celsius/second (e.g., 0.1-1.0 degrees Celsius/second, 0.25 to 2.5 degrees Celsius/second, 0.5-2.0 degrees Celsius/second, 1.0-3.0 degrees Celsius/second, 1.5-4.0 degree Celsius/second, 2.0-5.0 degrees Celsius/second). The time of treatment can range from between about 10 seconds and about 1 hour (e.g., from 10 seconds to 1 minute, 1 minute to 5 minutes, from 5 minutes to 10 minutes, from 5 minutes to 20 minutes, from 8 minutes to 15 minutes, from 10 minutes to 20 minutes, from 15 minutes to 30 minutes, from 20 minutes to 40 minutes, from 30 minutes to 1 hour, from 45 minutes to 1 hour, or overlapping ranges thereof). Pulsed energy can be delivered as an alternative to or in sequence with continuous energy. For radiofrequency energy, the energy applied by the RF generator can range from between about 350 kHz and about 650 kHz (e.g., from 400 kHz to 600 kHz, from 350 kHz to 500 kHz, from 450 kHz to 550 kHz, from 500 kHz to 650 kHz, overlapping ranges thereof, or any value within the recited ranges, such as 450 kHz±5 kHz, 475 kHz±5 kHz, 487 kHz±5 kHz). The power of the radiofrequency energy generated by the RF generator can range from between about 5 W and about 30 W (e.g., from 5 W to 15 W, from 5 W to 20 W, from 8 W to 12 W, from 10 W to 25 W, from 15 W to 25 W, from 20 W to 30 W, from 8 W to 24 W, and overlapping ranges thereof, or any value within the recited ranges).

Although described primarily in connection with procedures for the treatment within the spine, the systems and methods of the present invention can also be used for radiofrequency tissue ablation procedures intended to treat ailments or conditions other than those associated with the spine or back pain. The procedures can involve ablation of nerves outside of bones but related to the spine (e.g., sacroiliac joints, facet joints, etc.). Target treatment locations within bones other than vertebral bodies may also be accessed. For example, target treatment locations within a humerus, radius, femur, tibia, calcaneus, tarsal bones, hips, knees, phalanges, and/or other orthopedic targets can be accessed. The ablation procedures can include, for example, ablation of nerves within or surrounding other bones other than the vertebral column, cardiac tissue ablation for treatment of atrial fibrillation or other abnormal heart rhythms irregularities, tumor ablation at any location within the body (e.g., within bones, lungs, breasts, thyroids, livers, or other organs or tissues), peripheral nerve ablation, pulmonary artery ablation, renal denervation procedures, uterine fibroid ablation, endometrial ablation, and/or the like. The systems and methods described herein may be used in connection with any radiofrequency procedure during which impedance (e.g., tissue impedance or impedance between two electrodes) is monitored.

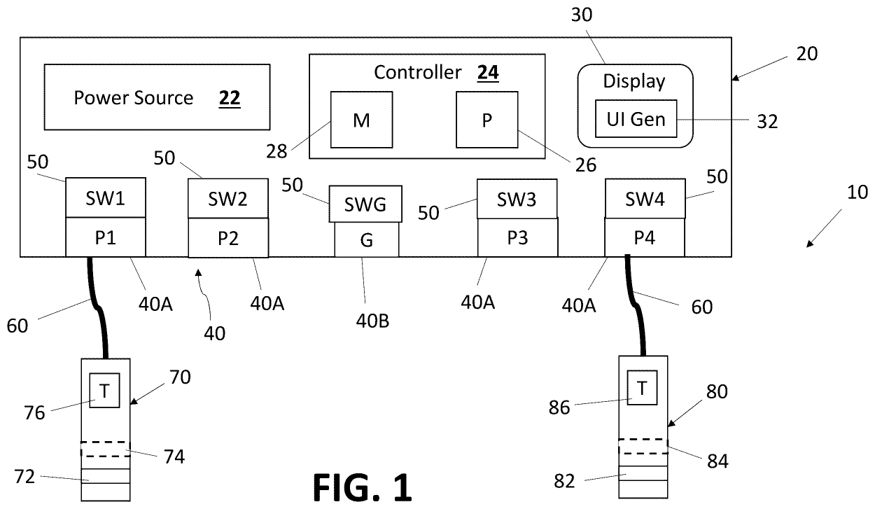

FIG. 1 is a schematic block diagram of a tissue ablation system 10 suitable for use with the present invention. The tissue ablation system includes a generator, such as a radiofrequency generator 20, that is coupled to one or more radiofrequency probes 70, 80. The RF generator 20 can be electrically and communicatively coupled to the RF probes 70, 80 via any suitable conduit 60. The conduits 60 can facilitate bidirectional communication of electrical signals and data or instructions between the RF generator 20 and the RF probes 70, 80. For example, the conduits 60 can include one or more electrical wires or lines (not shown) suitable for conveying the electrical signals. The generator can also be any other type of energy source suitable for supplying ablative energy to the probes. According to one embodiment, the illustrated RF generator 20 can include a power source 22 that can be configured to generate and supply radiofrequency power or energy to the RF probes 70, 80 via the conduits 60 at a desired frequency and power level. The RF generator 20 can also include a controller 24 that includes for example a processor (e.g., CPU) 26 suitable for processing and executing applications and instructions that are stored in a memory element 28. The processor 26 can be any suitable type of processor, such as a special purpose processor, and the memory element 28 can be any suitable type of memory. The processor 26 can be implemented in hardware, software, firmware, or any suitable combination thereof, and can be configured to execute program instructions stored in the memory element 28. The processor 26 and the memory element 28 are shown herein as forming part of the controller 24, but one of ordinary skill in the art will readily recognize that the processor 26 and the memory 28 can form part of other components of the RF generator 20. For the sake of simplicity, only one processor 26 and memory element 28 are shown, although multiple processors and memory elements can also be employed in the RF generator 20. The controller 24 can be configured as described herein, and at least is configured to monitor temperature, power, and/or impedance in connection with a tissue ablation procedure performed by the tissue ablation system 10. The RF generator 20 can further include a user interface or display element 30 suitable for displaying information or instructions to a user (e.g., surgeon) via a series of user interfaces or for allowing the user to provide instructions to the RF generator. As such, the display element 30 can employ a user interface generator 32 for generating the user interfaces based on programmatic instructions stored in the memory element 28 and processed by the processor 28 or by a processor forming part of the display element 30. The display element 30 can be configured to display information to the user. For example, during startup and use, the current status of the RF generator 20 and energy delivery or treatment parameters can be displayed on the display element 30 via one or more user interfaces. During energy delivery, the display element 30 can be configured to display desired treatment time, remaining treatment time, temperature, impedance, and power information (alphanumerically and/or graphically). For example, graphical representations of power vs. time and impedance vs. time can be displayed. The display element 30 can also be separate from the RF generator 20 and need not form part of the generator. Although not shown, the tissue ablation system 10 can also employ additional accessory devices, such as suitable user input devices including a keyboard, mouse, trackpad, voice-activated input device, and the like.

The illustrated RF generator 20 can also have formed therein a plurality or series of connector ports 40 that allow the RF probes 70, 80 to be coupled to the generator. The RF generator 20 can have any selected number of connector ports 40. In the illustrated embodiment, the RF generator 20 includes four probe connector ports 40A and a ground connector port 40B. The connector ports 40 can have any selected size, shape, and configuration. According to one embodiment, the connector ports 40 can employ a series of electrical pins that connect to RF probes and a ground pad and allow electrical energy and data to be exchanged therebetween. For example, a first electrical pin (or multiple electrical pins) can be coupled to the RF probe so as to deliver RF power to an energy delivering electrode when placed thereon and a second electrical pin can communicate with a return electrode when placed thereon. A third electrical pin can be employed to communicate with a temperature sensor in the RF probe and a fourth electrical pin can be used to exchange data between the RF generator and the RF probe. Those of ordinary skill in the art will readily recognize that any suitable type (e.g., Lemo or Din type connectors), arrangement, and number of electrical connectors can be employed in the connector ports 40.

The illustrated RF generator 20 can also employ a switching element module or a series of discrete switching elements 50 that can be used to help control the generation, modulation, and delivery of RF energy. The switching elements 50 help ensure the precise delivery of energy to the target tissue via the RF probes while maintaining system safety and functionality. The switching elements 50 can also control the RF energy delivered to the active energy delivering electrode of the RF probe through an associated connector port 40. For the sake of simplicity, a switching element 50 is shown coupled to and disposed in electrical communication with each illustrated connector port 40. Those of ordinary skill in the art will readily recognize that a separate switching element module that is arranged in electrical communication with each of the connector ports 40 can also be employed. The illustrated switching elements 50 can also be positioned and arranged to manage the activation of circuits connected to sensors in the RF probes 70, 80, such as temperature sensors or impedance measurement systems, and to manage the activation of the ground connector port 40B. The switching elements 50 can also be arranged to route sensor signals to specific monitoring circuits in the controller 24 for processing. The switching elements 50 can also serve to verify proper grounding or return electrode connection before enabling RF output to ensure patient safety. The switching elements 50 can be any suitable type of switching element, such as MOSFETs, IGBTs, Pin diodes, mechanical and solid state relays, and the like.

The illustrated tissue ablation system 10 can also employ one or more RF probes. For the sake of simplicity and for purposes of explanation, a pair of RF probes 70, 80 are shown connected to the RF generator 20. The RF probes 70, 80 can be the same or can be different depending upon the selected type of surgical procedure being performed and/or the purpose and function of the RF probe. The RF probe 70 has a main body having an outer surface having one or more electrodes mounted thereon. In a bipolar RF probe arrangement, the RF probe 70 can have a first active energy delivering electrode 72 and a return electrode 74 disposed on an outer surface of the main body. The probe can also include a temperature sensor 76 for sensing or measuring temperature at the surgical site. The electrodes and the temperature sensor 76 can be coupled to the RF generator 20 via electrical leads that pass through the conduit 60. If the RF probe 70 is constructed as a monopolar device, then the probe can include a single active electrode 72, which can function as the active energy delivering electrode or as the return electrode. In this type of arrangement, the second electrode 74 (in dashed lines) is either inactivated or not present. Similarly, the RF probe 80 has a main body having an outer surface having one or more electrodes mounted thereon. In a bipolar RF probe arrangement, the RF probe 80 can also have a first active energy delivering electrode 82 and a return electrode 84. The probe can also include a temperature sensor 86. The electrodes and the sensor can also be coupled to the RF generator 20 via electrical leads that pass through the conduit 60. If the RF probe 80 is constructed as a monopolar device, then the probe can also include a single active electrode 82, which can function as the active energy delivering electrode or as the return electrode. In this type of arrangement, the second electrode 84 is either inactivated or not present. Each RF probe that is coupled to a connector port 40 of the RF generator 20 forms in essence a channel. As such, multiple RF probes coupled to multiple connector ports 40 of the RF generator 20 can form multiple channels.

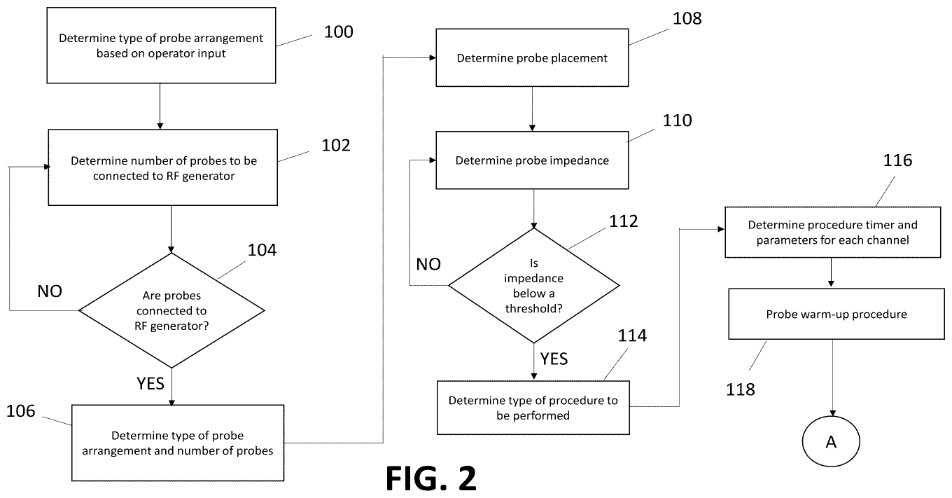

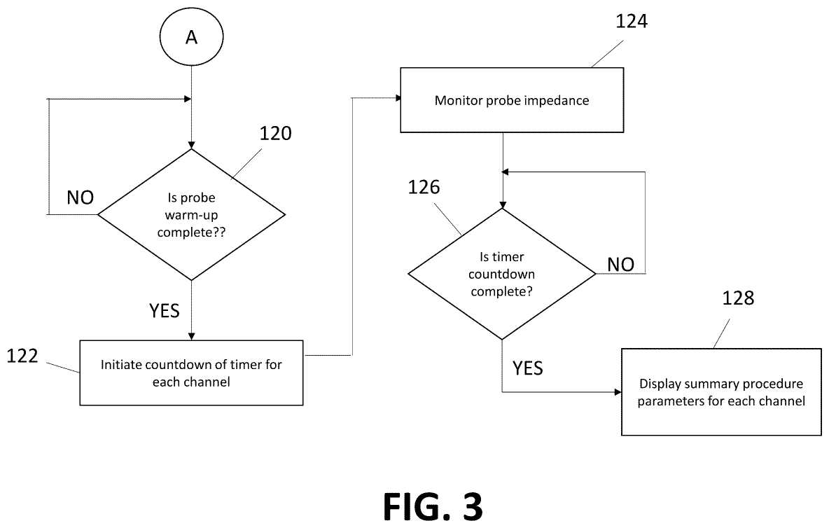

FIGS. 2 and 3 are schematic flow chart diagrams illustrating the operational modes implemented by the controller 24 when the RF generator 20 is used during an ablation procedure. In use, the operator turns on the RF generator 20 and then the operator can select the type of procedure to be performed and number of electrodes used, and indicate if the RF probes will be used in a monopolar arrangement or configuration or a bipolar arrangement or configuration. The controller 24 then initially determines, according to one operational mode, the type and number of devices that are attached to the connector ports 40. Specifically, the controller 24 can automatically determine if a ground pad (not shown) is coupled to the ground connector port 40B and/or if one or more RF probes 70, 80 are connected to one or more of the connector ports 40A, step 100. The controller 24 can also determine the number of RF probes 70, 80 that should be connected to the RF generator 20. The controller 24 then determines if the RF probes are properly connected to the connector ports 40, step 104. If the probes are not connected to the connector ports, the controller 24 waits for the operator to properly connect the probes to the connector ports 40. If the probes are properly connected, then the controller 24 determines the type of RF probe configuration, such as the number of probes connected to the connector ports 40 and whether the RF probes 70, 80 are being used in a monopolar or bipolar configuration, step 106. The controller 24 can also be programmed and configured to display via the display element 30 information associated with multiple channels via suitably configured user interfaces. For example, the display element 30 can display information associated with channel 1 (e.g., a first RF probe) and with channel 2 (e.g., a second RF probe), if multiple RF probes are connected to the RF generator 20.

Once the RF probes 70, 80 are connected to the RF generator 20, the controller 24 then determines, according to another operational mode, the location or position of the RF probe relative to the patient by sensing or determining the temperature of the RF probe and then comparing the temperature to a prestored or threshold temperature, step 108. If the measured probe temperature is below the threshold temperature, then the controller 24 determines that the RF probe is not placed within the patient. If the temperature is above the threshold temperature value, then the controller 24 determines that the probe has indeed been inserted or placed within the patient. According to one embodiment, the threshold temperature value can be in a range that represents ambient environment, such as between about 25° C. and about 40° C., and can be optionally set at 33° C. The controller 24 can then determine, according to still another operational mode, the impedance of one or more of the attached RF probes via known impedance measurement techniques, such as by sensing and measuring the voltage and current of the RF probe and then determining based thereon the probe impedance, step 110. The measured probe impedance indicates if the electrodes of the RF probe are exposed to tissue within the patient or whether the electrodes of the probe are still housed or covered within the cannula portion of the introducer assembly. According to one embodiment, the controller 24 can determine the impedance of the RF probe and then compare the measured impedance to a threshold impedance value, step 112. If the measured impedance is above the threshold impedance value, then the controller 24 determines that the electrodes of the RF probe are within the cannula and the controller then continues to measure the probe impedance to determine when the impedance value falls below the threshold impedance value. When the impedance value is below the threshold impedance value, then the controller 24 determines that the electrodes are properly exposed and can next allow operator input regarding the type of procedure to be performed. The impedance threshold value can be any suitable impedance value or impedance range and can be set based on the electrode design and the specific anatomy of the patient. According to one embodiment, the threshold impedance value can be between about 1000 ohms and about 3500 ohms, and optionally can be set at about 1500 ohms. The controller 24 of the RF generator 20 can be configured or programmed to perform an express assessment of the functionalities of the operational modes shown in steps 100-112. Specifically, the controller 24 can automatically determine, in sequence. the foregoing operational modes, including whether the RF probes are connected to the connector ports, the number of RF probes connected, the type of probe arrangement, the location of the RF probe based on measured temperature, and the probe impedance, without operator involvement. Further, the ability to determine automatically the foregoing operational functionalities and parameters easily and quickly, and in sequence, enables the RF generator 20 to cycle through the operational parameters associated with the set-up of the medical procedure that is to be performed by the operator without requiring operator input or intervention.

The RF generator 20 can also enable the operator to input selected procedure-related data or information by generating with the user interface generator 32 one or more selected user interfaces that can be displayed on the display element 30. The operator can select the type of procedure to be performed on the patient via the generated user interface. For example, and according to one example embodiment, the operator can select between a targeted procedure of a first selected time duration, such as about 7 minutes, and a standard procedure of a second selected time duration, such as about 15 minutes, via actionable soft buttons employed in the user interface, step 114. Optionally, the user can also select or specify a specific ablation temperature employed by the RF probe. The ablation temperatures correspond to specific ablation sizes. For example, the operator can select a 75° C. ablation temperature that can correspond to a 5 mm radial ablation size or zone, or the operator can select an 85° C. ablation temperature that can correspond to a 6 mm radial ablation size or zone. The ablation procedure time and temperature can be selected for each channel that is being employed during the ablation procedure, step 116. Once the user selection information is received, the user interface generator 32 can generate a user interface that displays a timer element corresponding to the selected procedure duration and can optionally display one or more parameters associated with the RF generator and with the RF probe. For example, the parameters can include probe impedance, probe temperature, power supplied to the probe, voltage, current, procedure time, time remaining in the procedure, and the like. Once the requisite user selection information is received and processed by the controller 24, then the controller can optionally initiate a probe warm-up procedure, step 118. During the probe warm-up procedure, the controller 24 can automatically actuate the power source 22 to apply relatively low-level RF power to the RF probe to warm the probe prior to initiation of the ablation procedure. For example, the controller 24 can initiate, according to another operational mode, warming of the RF probe for a selected period of time between about 30 seconds and about 5 minutes. The RF probe can be warmed to a suitable temperature to avoid thermal shock to the probe. As such, the warm-up temperature for the RF probe can be in the range of about 36° C. to about 43° C., which typically aligns with body temperature to avoid thermal shock during insertion. The warm-up time can be dependent upon the type of RF generator and RF probe, environmental conditions (e.g., ambient temperature), the type and number of system diagnostic or calibration steps integrated into the warm-up procedure, and the like. The controller 24 can monitor the probe temperature during the warm-up procedure to determine whether the probe temperature reaches or exceeds a selected warm-up stage probe temperature threshold value, step 120. Once the probe temperature reaches or exceeds the temperature threshold value, then the ablation procedure can be started or initiated and the timer can start to decrement, step 122. Further, the user interface displayed on the display element 30 can display selected parameter information, such as the real time probe impedance, real-time probe temperature, and the RF power (in Watts) supplied to the RF probe by the RF generator 20.

During the ablation procedure, the timer decrements to indicate the time remaining in the ablation procedure, step 122. The controller 24 also monitors and determines the probe impedance during the procedure to determine if an impedance remediation or correction procedure needs to be implemented, step 124. Similarly, the probe temperature can also be monitored. The controller 24 also tracks or monitors the procedure time, step 126. When the procedure is completed, then the user interface generator can generate a user interface that displays selected summary information of selected procedure-related parameters via any suitable report format, step 128. For example, the user interface can display summary information associated with the probe temperature, probe impedance, and power. The report can have any suitable format, provided that the report shows the parameter information during selected time frames or periods associated with the entire ablation procedure. According to another embodiment, the report format can include parameter values at selected time points or portions of the ablation procedure. The controller 24 can also provide information to the display element 30 such that the user interface generator can generate one or more suitable user interfaces for displaying the procedure-related information associated with each channel during one or more of the operational modes.

Examples of the user interfaces that can be generated by the user interface generator 32 for subsequent display on the display element 30 during the various operational modes are shown for example in Appendix 1. Further, example state diagrams of the RF generator 20 are shown in Appendix 2.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A tissue ablation system, comprising:
a radiofrequency generator having a power source, a controller operatively coupled to the power source and having a processor and a memory element, a plurality of connection ports, and a display element, and
a first radiofrequency probe electrically coupled to the radiofrequency generator to form a first channel and having at least one first electrode and a first temperature sensor,
wherein the power source is configured to deliver radiofrequency power to the at least one first electrode of the first radiofrequency probe, the radiofrequency power being sufficient to provide controlled heating of tissue of a patient surrounding the at least one first electrode during an ablation procedure, wherein the processor is configured to execute program instructions stored in the memory element such that, when executed, cause the processor to:
(a) determine if the first radiofrequency probe is electrically coupled to the radiofrequency generator,
(b) determine a type of electrode arrangement of the first radiofrequency probe,
(c) determine a placement position of the first radiofrequency probe relative to the patient,
(d) determine a probe impedance of the first radiofrequency probe to determine if the at least one first electrode is exposed to the tissue,
(e) based on user selection data, determine a duration of the ablation procedure with the first radiofrequency probe,
(f) perform a warm-up procedure to warm up the first radiofrequency probe,
(g) initiate the ablation procedure by supplying power to the first radiofrequency probe,
(h) display a time remaining in the ablation procedure with the first radiofrequency probe on the display element, and
(i) upon completion of the ablation procedure with the first radiofrequency probe, display on the display element a summary of selected parameters associated with the first radiofrequency probe, wherein the selected parameters include an impedance of the first radiofrequency probe during the ablation procedure, a temperature of the first radiofrequency probe during the ablation procedure, and a power supplied by the power source to the first radiofrequency probe during the ablation procedure.

2. The tissue ablation system of claim 1, wherein the processor is further configured to automatically perform (a)-(d), in sequence.

3. The tissue ablation system of claim 1, wherein the processor is further configured to automatically perform any combination of two or more of (a)-(d).

4. The tissue ablation system of claim 1, wherein the processor is configured to determine the placement position of the first radiofrequency probe by processing temperature data from the first temperature sensor and comparing the temperature data to a threshold temperature to determine location.

5. The tissue ablation system of claim 1, wherein the processor is further configured to determine a probe impedance of the first radiofrequency probe by comparing the probe impedance with a threshold probe impedance value and then determine that the at least one first electrode is exposed to the tissue when the probe impedance is below the threshold probe impedance value.

6. The tissue ablation system of claim 1, further comprising a second radiofrequency probe electrically coupled to the radiofrequency generator to form a second channel and having at least one second electrode and a second temperature sensor.

7. The tissue ablation system of claim 6, wherein the processor is further configured to independently:
(j) determine if the second radiofrequency probe is electrically coupled to the radiofrequency generator,
(k) determine a type of electrode arrangement of the second radiofrequency probe,
(l) determine a placement position of the second radiofrequency probe relative to the patient, and
(m) determine a probe impedance of the second radiofrequency probe to determine if the at least one second electrode is exposed to the tissue of the patient.

8. The tissue ablation system of claim 7, wherein the processor is further configured to:
based on user selection data, determine a duration of the ablation procedure with the second radiofrequency probe,
perform a warm-up procedure to warm up the second radiofrequency probe,
initiate the ablation procedure by supplying power to the second radiofrequency probe,
display a time remaining in the ablation procedure with the second radiofrequency probe on the display element, and
upon completion of the ablation procedure with the second radiofrequency probe, display a summary of selected parameters associated with the second radiofrequency probe, wherein the selected parameters include an impedance of the second radiofrequency probe during the ablation procedure, a temperature of the second radiofrequency probe during the ablation procedure, and the power supplied by the power source to the second radiofrequency probe during the ablation procedure.

9. The tissue ablation system of claim 6, wherein the processor is further configured to display information associated with the first channel and the second channel on the display element.

10. The tissue ablation system of claim 6, wherein the processor is further configured to display information associated with the first channel and the second channel individually or simultaneously on the display element.

11. The tissue ablation system of claim 1, wherein the plurality of connector ports comprises at least one ground connector port and a plurality of probe connector ports.

12. A tissue ablation system, comprising:
a radiofrequency generator having a power source, a controller operatively coupled to the power source and having a processor and a memory element, a plurality of connection ports, and a display element, and
a first radiofrequency probe electrically coupled to the radiofrequency generator to form a first channel and having at least one first electrode and a first temperature sensor,
wherein the power source is configured to deliver radiofrequency power to the at least one first electrode of the first radiofrequency probe, the radiofrequency power being sufficient to provide controlled heating of tissue of a patient surrounding the at least one first electrode during an ablation procedure, wherein the processor is configured to execute program instructions stored in the memory element such that, when executed, cause the processor to automatically perform, in sequence:
(a) determine if the first radiofrequency probe is electrically coupled to the radiofrequency generator,
(b) determine a type of electrode arrangement of the first radiofrequency probe,
(c) determine a placement position of the first radiofrequency probe relative to the patient, and
(d) determine a probe impedance of the first radiofrequency probe to determine if the at least one first electrode is exposed to the tissue.

13. The tissue ablation system of claim 12, wherein the processor is configured to determine the placement position of the first radiofrequency probe by processing temperature data from the first temperature sensor and comparing the temperature data to a threshold temperature to determine location.

14. The tissue ablation system of claim 12, wherein the processor is further configured to determine a probe impedance of the first radiofrequency probe by comparing the probe impedance with a threshold probe impedance value and then determine that the at least one first electrode is exposed to the tissue when the probe impedance is below the threshold probe impedance value.

15. A computer-implemented method of ablating tissue in an ablation procedure using a tissue ablation system, wherein the tissue ablation system includes:
a radiofrequency generator having a power source, a controller operatively coupled to the power source and having a processor and a memory element, a plurality of connection ports, and a display element, and
a first radiofrequency probe electrically coupled to the radiofrequency generator to form a first channel and having at least one first electrode and a first temperature sensor,
wherein the power source is configured to deliver radiofrequency power to the at least one first electrode of the first radiofrequency probe, the radiofrequency power being sufficient to provide controlled heating of tissue of a patient surrounding the at least one first electrode during an ablation procedure, the method comprising configuring the processor to execute program instructions stored in the memory element such that, when executed, cause the processor to:
(a) determine if the first radiofrequency probe is electrically coupled to the radiofrequency generator,
(b) determine a type of electrode arrangement of the first radiofrequency probe,
(c) determine a placement position of the first radiofrequency probe relative to the patient,
(d) determine a probe impedance of the first radiofrequency probe to determine if the at least one first electrode is exposed to the tissue,
(e) based on user selection data, determine a duration of the ablation procedure with the first radiofrequency probe,
(f) perform a warm-up procedure to warm up the first radiofrequency probe,
(g) initiate the ablation procedure by supplying power to the first radiofrequency probe,
(h) display a time remaining in the ablation procedure with the first radiofrequency probe on the display element, and
(i) upon completion of the ablation procedure with the first radiofrequency probe, display on the display element a summary of selected parameters associated with the first radiofrequency probe, wherein the selected parameters include an impedance of the first radiofrequency probe during the ablation procedure, a temperature of the first radiofrequency probe during the ablation procedure, and a power supplied by the power source to the first radiofrequency probe during the ablation procedure.

16. The computer-implemented method of claim 15, further comprising configuring the processor to automatically perform steps (a)-(d), in sequence.

17. The computer-implemented method of claim 15, further comprising configuring the processor to determine the placement position of the first radiofrequency probe by processing temperature data from the first temperature sensor and comparing the temperature data to a threshold temperature to determine location.

18. The computer-implemented method of claim 15, further comprising configuring the processor to determine a probe impedance of the first radiofrequency probe by comparing the probe impedance with a threshold probe impedance value and then determining that the at least one first electrode is exposed to the tissue when the probe impedance is below the threshold probe impedance value.

19. The computer-implemented method of claim 15, further comprising coupling a second radiofrequency probe electrically to the radiofrequency generator to form a second channel and having at least one second electrode and a second temperature sensor.

20. The computer-implemented method of claim 19, wherein the processor is further configured to independently:
(j) determine if the second radiofrequency probe is electrically coupled to the radiofrequency generator,
(k) determine a type of electrode arrangement of the second radiofrequency probe,
(l) determine a placement position of the second radiofrequency probe relative to the patient, and
(m) determine a probe impedance of the second radiofrequency probe to determine if the at least one second electrode is exposed to the tissue of the patient.
